(19) United States
(12) Patent Application Publication
(10) Pub. No.: US 2022/0218274 A1
(43) Pub. Date: Jul. 14, 2022

(54) Methods for Estimating Key Phenotypic Traits for Obstructive Sleep Apnea and Simplified Clinical Tools to Direct Targeted Therapy

(71) Applicant: Flinders University, South Australia (AU)

(72) Inventors: Danny J. Eckert, Randwick, New South Wales (AU); Ritaban Dutta, Randwick, New South Wales (AU)

(21) Appl. No.: 17/613,041

(22) PCT Filed: May 22, 2020
(86) PCT No.: PCT/AU2020/050506

§ 371 Date: Nov. 20, 2021

(30) Foreign Application Priority Data
May 22, 2019 (AU) 2019901736

(51) Int. Cl. A61B 5/00; A61B 5/1455; A61B 5/08; A61B 5/107; G06N 20/00; G06N 5/00; G16H 20/40; G16H 50/20; A61B 5/087; A61F 5/56; G06N 7/00; G06N 3/02

(52) U.S. Cl. CPC A61B 5/4818; G06N 3/02; A61B 5/0826; A61B 5/107; A61B 5/4809; A61B 5/4812; A61B 5/4815; A61B 5/4872; A61B 5/7264; A61B 5/7275; G06N 20/00; G06N 5/003; G16H 20/40; G16H 50/20; A61B 5/0871; A61F 5/566; G06N 7/005; A61B 5/14551

(57) ABSTRACT

A method of predicting the likelihood of responding to one or more obstructive sleep apnea (OSA) treatments for obstructive sleep apnea (OSA) of a candidate subject, the method including the steps of: (a) measuring a first series of polysomnography and anthropometric parameters for at least a collection of OSA sufferers; (b) correlating the parameters with at least one of the corresponding upper-airway collapsibility (Pcrit), arousal threshold, loop gain and pharyngeal muscle responsiveness measurement of each subject; (c) determining a corresponding description structure describing the correlation of step (d); and (d) utilising the corresponding description structure, in conjunction with a series of polysomnography and anthropometric parameters measured for the candidate subject to predict the likelihood of responding to one or more obstructive sleep apnea (OSA) treatments to thereby provide a clinical decision diagnostic tool for obstructive sleep apnea.

### FIELD OF THE INVENTION

The present invention provides for systems and methods for the estimation of key phenotypic traits (also referred to as “endotypes”) for obstructive sleep apnea and simplified clinical tools to direct targeted therapy.

### REFERENCES

1. Eckert D J. Phenotypic approaches to obstructive sleep apnoea—New pathways for targeted therapy. Sleep Med Rev 2018; 37:45-59.
2. Eckert D J, White D P, Jordan A S, Malhotra A, Wellman A. Defining phenotypic causes of obstructive sleep apnea. Identification of novel therapeutic targets. American journal of respiratory and critical care medicine 2013; 188:996-1004.
3. Edwards B A, Sands S A, Owens R L, et al. The Combination of Supplemental Oxygen and a Hypnotic Markedly Improves Obstructive Sleep Apnea in Patients with a Mild to Moderate Upper Airway Collapsibility. Sleep 2016; 39:1973-83.
4. Edwards B A, Sands S A, Eckert D J, et al. Acetazolamide improves loop gain but not the other physiological traits causing obstructive sleep apnoea. J Physiol 2012; 590:1199-211.
5. Taranto-Montemurro L, Sands S A, Edwards B A, et al. Desipramine improves upper airway collapsibility and reduces OSA severity in patients with minimal muscle compensation. Eur Respir J 2016; 48:1340-50.
6. Eckert D J, Owens R L, Kehlmann G B, et al. Eszopiclone increases the respiratory arousal threshold and lowers the apnoea/hypopnoea index in obstructive sleep apnoea patients with a low arousal threshold [Accelerated Publication]. Clin Sci (Lond) 2011; 120:505-14.
7. Wellman A, Malhotra A, Jordan A S, Stevenson K E, Gautam S, White D P. Effect of oxygen in obstructive sleep apnea: role of loop gain. Respir Physiol Neurobiol 2008; 162:144-51.
8. Messineo L, Magri R, Corda L, Pini L, Taranto-Montemurro L, Tantucci C. Phenotyping-based treatment improves obstructive sleep apnea symptoms and severity: a pilot study. Sleep Breath 2017; 21:861-8.
9. Sands S A, Edwards B A, Terrill P I, et al. Identifying obstructive sleep apnoea patients responsive to supplemental oxygen therapy. Eur Respir J 2018; 52.
10. Carberry J C, Amatoury J, Eckert D J. Personalized Management Approach for OSA. Chest 2018; 153:744-55.
11. Owens R L, Edwards B A, Eckert D J, et al. An Integrative Model of Physiological Traits Can be Used to Predict Obstructive Sleep Apnea and Response to Non Positive Airway Pressure Therapy. Sleep 2015; 38:961-70.
12. Azarbarzin A, Sands S A, Taranto-Montemurro L, et al. Estimation of Pharyngeal Collapsibility During Sleep by Peak Inspiratory Airflow. Sleep 2017; 40.
13. Landry S A, Joosten S A, Eckert D J, et al. Therapeutic CPAP Level Predicts Upper Airway Collapsibility in Patients With Obstructive Sleep Apnea. Sleep 2017; 40.
14. Sands S A, Edwards B A, Terrill P I, et al. Phenotyping Pharyngeal Pathophysiology using Polysomnography in Patients with Obstructive Sleep Apnea. Am J Respir Crit Care Med 2018; 197:1187-97.
15. Osman A M, Carberry J C, Burke P G R, Toson B, Grunstein R R, Eckert D J. Upper airway collapsibility measured using a simple wakefulness test closely relates to the pharyngeal critical closing pressure (Pcrit) during sleep in obstructive sleep apnea. Sleep 2019 Jul. 8; 42(7). pii: zsz080. doi: 10.1093/sleep/zsz080.
16. Orr J E, Sands S A, Edwards B A, et al. Measuring Loop Gain via Home Sleep Testing in Patients with Obstructive Sleep Apnea. American journal of respiratory and critical care medicine 2018; 197:1353-5.
17. Messineo L, Taranto-Montemurro L, Azarbarzin A, et al. Breath-holding as a means to estimate the loop gain contribution to obstructive sleep apnoea. J Physiol 2018; 596:4043-56.
18. Terrill P I, Edwards B A, Nemati S, et al. Quantifying the ventilatory control contribution to sleep apnoea using polysomnography. Eur. Respir. J. 2015; 45:408-18.
19. Edwards B A, Eckert D J, McSharry D G, et al. Clinical predictors of the respiratory arousal threshold in patients with obstructive sleep apnea. American journal of respiratory and critical care medicine 2014; 190:1293-300.
20. Sands S A, Terrill P I, Edwards B A, et al. Quantifying the Arousal Threshold Using Polysomnography in Obstructive Sleep Apnea. Sleep 2018; 41.
21. Carberry J C, Jordan A S, White D P, Wellman A, Eckert D J. Upper airway collapsibility (Pcrit) and pharyngeal dilator muscle activity are sleep-stage dependent. Sleep 2016; 39:511-21.

### BACKGROUND OF THE INVENTION

Any discussion of the background art throughout the specification should in no way be considered as an admission that such art is widely known or forms part of common general knowledge in the field.

Obstructive sleep apnea (OSA) is an increasingly common sleep-related breathing disorder. It is characterized by repetitive upper airway narrowing (hypopnea) and closure (apnea) during sleep. If left untreated, OSA is associated with a range of adverse health outcomes including cardiovascular, metabolic and neurocognitive consequences. Continuous Positive Airway Pressure (CPAP) is administered as monotherapy according to a trial and error management approach in which the majority of patients diagnosed with OSA are first prescribed CPAP therapy. CPAP remains the first-line and most efficacious treatment for OSA. Delivered via nasal or oronasal mask, CPAP acts as a pneumatic splint to directly increase pharyngeal cross-sectional area and prevent collapse. CPAP also increases end-expiratory lung volume, which may indirectly improve airway function.

Although advances in CPAP technology and optimal mask selection can improve CPAP comfort and tolerance, failure rates are often high (>50% in some cases).

OSA has both anatomical and non-anatomical contributors or phenotypes/endotypes (1,2). Non-anatomical pathophysiological phenotypes such as a low respiratory arousal threshold, an oversensitive respiratory control system (high loop gain) and impaired upper-airway dilator muscle responsiveness during sleep are present in most people with OSA (1,2). In addition, some degree of anatomical impairment (i.e., narrow, crowded, collapsible airway) is a key feature of OSA for all patients (1,2).

Non-anatomical contributors play a prominent role in OSA pathogenesis for many patients. New and emerging therapies that target specific phenotypic or ‘treatable traits’ rather than a one size-fits-all approach show promise as an alternative to traditional therapies that focus on the anatomical problem. Studies show that 70% of OSA patients have impairment in one or more non-anatomical contributors (1,2).

Novel approaches to manipulate specific OSA phenotypes to reduce OSA severity have been performed in small physiology studies (3,9). These studies provide proof-of-concept support for a targeted phenotype based model to treat OSA (10,11). However, the gold standard methodology used to quantity OSA phenotypes is invasive and time-consuming. Thus, it is not practical for routine clinical care. Accordingly, a major objective to advance targeted therapy for OSA is to develop simplified techniques to accurately estimate the key phenotypic traits. In this regard there has been recent progress. For example, simple approaches to estimate airway collapsibility during sleep using standard polysomnography and CPAP titration data (12-14) have been developed. Wakefulness tests have also been developed to estimate upper airway collapsibility (15). In complimentary work, tools to simply quantify the non-anatomical traits that contribute to OSA such as loop gain (17-20), the respiratory arousal threshold (19, 20) and pharyngeal muscle effectiveness (21) have also been developed. However, to date, these approaches are yet to translate to clinical care. Thus, there remains an important need to develop simplified tools to estimate OSA phenotypes and to inform targeted therapy that can be readily integrated into clinical care.

The Pcrit, Arousal threshold, Loop gain, Muscle responsiveness (PALM) scale was developed based on the phenotyping concepts to inform tailored therapy (1,2). The goal of the PALM scale is to complement existing clinical measures (e.g. the apnea/hypopnea index [AHI], symptoms and comorbidities) to facilitate a more comprehensive personalized approach to inform treatment decisions in which patients are prescribed one or more therapies according to their specific underlying cause/s of OSA (1,2).

### SUMMARY OF THE INVENTION

It is an object of the invention, in its preferred form to provide for methods for estimating key phenotypic traits and simplified clinical tools for obstructive sleep apnea to direct targeted therapy.

In accordance with a first aspect of the present invention, there is provided a method of determining a likely cause of obstructive sleep apnea (OSA), the method including: (a) measuring a series of polysomnographic and anthropometric variables for the patient with OSA; (b) predicting the causes of the OSA for the patient; and (c) providing a targeted treatment for the patient based on the predicted causes of the OSA.

In some embodiments, the step (a) and (b) can include measuring a series of polysomnographic and anthropometric/clinical variables for the patient, and using the measured variables to estimate key OSA phenotypes for the patient.

In some embodiments, the step (a) includes measuring OSA phenotypes such as simple breathing, clinical and polysomnography measures, and step (b) includes using the measurements to identify likely responders to mandibular advancement therapy and combination therapy in people with OSA.

Specifically, there is provided an approach which takes in data from measurements of patients with OSA to predict their key causes of OSA so that targeted treatments for optimal patient outcomes can be recommended. The developed algorithmic approach found that standard polysomnographic and anthropometric variables could be used to accurately estimate key OSA phenotypes with a high degree of accuracy.

Based on the PALM classification of recorded patient data from a ‘cohort’ study, a predictive analytical model has been developed. There is proposed a predictive system designed to digest all possible polysomnographic inputs (e.g. AHI, oxygen saturation, arousal index etc.) and simple clinical measures (e.g. age, gender, BMI etc.) to predict a class of an unknown patient's phenotypic characterization as MILD/MODERATE/SEVERE—simple generalized classification solution for a widely complex data set and a very difficult to treat clinical condition. Predictions from this proposed model were validated, comparing the model prediction of OSA phenotypes to the gold standard quantification approach.

The first measure preferably can include a measure of each of upper-airway collapsibility (Pcrit), arousal threshold, loop gain and pharyngeal muscle responsiveness.

The prediction can be determined via principal component analysis of the first measure factors.

The principal component analysis can be combined with a decision tree learning system or a tailored, optimized algorithm to determine the degree of accuracy to be used to direct targeted therapy (Flow diagram FIG. 1).

In accordance with a further aspect of the present invention, there is provided a method of delivering targeted therapy for sleep apnea or related respiratory disorders, the method including the steps of collecting patient information on an individual patient including polysomnographic, anthropometric, demographic, clinical, physiological and breathing variables; Inputting the patient information into algorithmic tools to predict the accuracy with which the individual patient will respond to a variety of established and emerging treatments including combination therapies; Presenting the patient information and accuracy prediction to the patient so that they, in combination with their medical provider can make informed decisions about treatment selection; and Retaining and utilising the collected data from each patient for future use by the tools for further refinement and improvement of treatment prediction accuracy on an ongoing basis.

In accordance with a further aspect of the present invention, there is provided a method of determining a likely indicator for obstructive sleep apnea (OSA) of a human subject, the method including the steps of: (a) Measuring polysomnography and anthropometric parameters of a series of subjects including Gender (M/F), Age (years), BMI (kg/m²), Total AHI (events/h), Supine AHI (events/h), Nadir SaO₂ (%), Non-REM AHI (events/h), Supine non-REM AHI (events/h), REM AHI (events/h sleep), Arousal index (#/h sleep) and Fraction of hypopneas v. apneas; (b) determining the principal components of the parameters utilising a principal component analysis; (c) correlating the principal components with the upper-airway collapsibility (Pcrit), arousal threshold, loop gain and pharyngeal muscle responsiveness measurements of each subject; (d) utilising the correlated measurements to derive a data driven supervised machine learning structure describing the correlation; and (e) for a given human subject, measuring the subject's polysomnography and anthropometric parameters and utilising the machine learning structure to determine an estimate of the upper-airway collapsibility, arousal threshold, loop gain and pharyngeal muscle responsiveness of the human subject.

In accordance with a further aspect of the present invention, there is provided a method of predicting the likelihood of responding to one or more obstructive sleep apnea (OSA) treatments to be used as a clinical decision diagnostic tool of a candidate subject, the method including the steps of: (a) measuring a first series of polysomnography and anthropometric parameters for at least a collection of OSA sufferers; (b) correlating the parameters with at least one of the corresponding upper-airway collapsibility (Pcrit), arousal threshold, loop gain and pharyngeal muscle responsiveness measurement of each subject; (c) determining a corresponding description structure describing the correlation of step (d); and (d) utilising the corresponding description structure, in conjunction with a series of polysomnography and anthropometric parameters measured for the candidate subject to determine the likelihood of responding to one or more obstructive sleep apnea (OSA) treatments to be used as a clinical decision diagnostic tool for obstructive sleep apnea.

In some embodiments the correlating in step (b) includes determining a principal component analysis of the measured parameters of step (a). In some embodiments, the correlation in step (b) includes applying multivariate principal component analyses (PCA) to the polysomnography and anthropometric parameters to determine the principal components of the parameters for at least the collection of OSA sufferers. In some embodiments, the corresponding descriptive structure comprises a decision tree learner. In some embodiments, a decision tree is determined for each of upper-airway collapsibility (Pcrit), arousal threshold, loop gain and pharyngeal muscle responsiveness measurement.

In some embodiments, the series of polysomnography and anthropometric parameters includes at least one of: age, BMI, total AHI, supine AHI, nadir SaO₂, non-REM AHI, supine non-REM AHI, REM AHI, arousal index, sleep efficiency and the fraction of hypopneas vs. apneas. In some embodiments, the OSA comprises at least one of: 1) upper-airway collapsibility (Pcrit), 2) arousal threshold, 3) loop gain and 4) pharyngeal muscle responsiveness. In some embodiments, the step (a) includes measuring OSA sufferers and non-sufferers.

### BRIEF DESCRIPTION OF THE DRAWINGS

Embodiments of the invention will now be described, by way of example only, with reference to the accompanying drawings in which:

FIG. 1 illustrates a flow chart of the overall process of the embodiment.

FIG. 2 illustrates examples PC Analysis on OSA phenotypes from 52 participants which indicates Linear Separability and confirms suitability for predictive analysis.

FIG. 3 illustrates a Decision Tree for Loop Gain Prediction. This figure (as per the other decision tree figures below) shows the internal workings of the method. Gini impurity defined as “gini” is a measure of how often a randomly chosen element from the set would be incorrectly labelled if it was randomly labelled according to the distribution of labels in the subset. It reaches its minimum (zero) when all cases in the node fall into a single target category. At that stage, the ‘root node’ converge into a ‘leaf node’ demarking the end of a decision branch, or ‘root node’ itself to become a ‘leaf node’ with ‘gini=0’. Left indicates that the criterion is met (true) whereas right indicates that the criterion is not met (false).

FIG. 4 illustrates a Decision Tree for PCRIT Prediction

FIG. 5 illustrates a Decision Tree for Arousal Threshold Prediction

FIG. 6 illustrates a Decision Tree for Muscle Responsiveness Prediction

FIG. 7 shows the final decision tree for Total AHI prediction.

FIG. 8 shows the final decision tree for non-rapid eye movement (REM) AHI prediction.

FIG. 9 shows the final decision tree for REM AHI prediction.

FIG. 10 to FIG. 13 illustrate various 3D visualisations for PC Analysis (and the proportion of variance explained by each PC highlighted next to each axis) and MAS therapy responder (triangles) versus non-responder (circles) characteristics for the 44 participants studied on MAS therapy according to 4 common definitions of treatment success (FIG. 10. AHI on MAS less than 5 events per hour sleep, FIG. 11. AHI on MAS less than 10 events per hour sleep, FIG. 12. 50% reduction in total AHI and FIG. 13. 50% reduction in total AHI to less than 20 events per hour sleep).

FIG. 14 to FIG. 17 illustrate various visualisations of trained trees for MAS therapy prediction according to 4 common definitions of treatment success (FIG. 11. AHI on MAS less than 5 events per hour sleep, FIG. 12. AHI on MAS less than 10 events per hour sleep, FIG. 13. 50% reduction in total AHI and FIG. 14. 50% reduction in total AHI to less than 20 events per hour sleep).

FIG. 18 illustrates various responder plots (according to 4 common definitions of treatment success) against principal components for combination therapy (MAS and Oral and Nasal Valves);

FIG. 19 illustrates a trained tree for total AHI less than 5 events per hour sleep (MAS and Oral and Nasal Valves combination therapy);

FIG. 20 illustrates a trained tree for total AHI less than 10 events per hour sleep (MAS and Oral and Nasal Valves combination therapy);

FIG. 21 illustrates a trained tree for a 50% reduction in total AHI (MAS and Oral and Nasal Valves combination therapy); and

FIG. 22 illustrates a trained tree for a 50% reduction in total AHI to less than 20 events per hour sleep (MAS and Oral and Nasal Valves combination therapy);

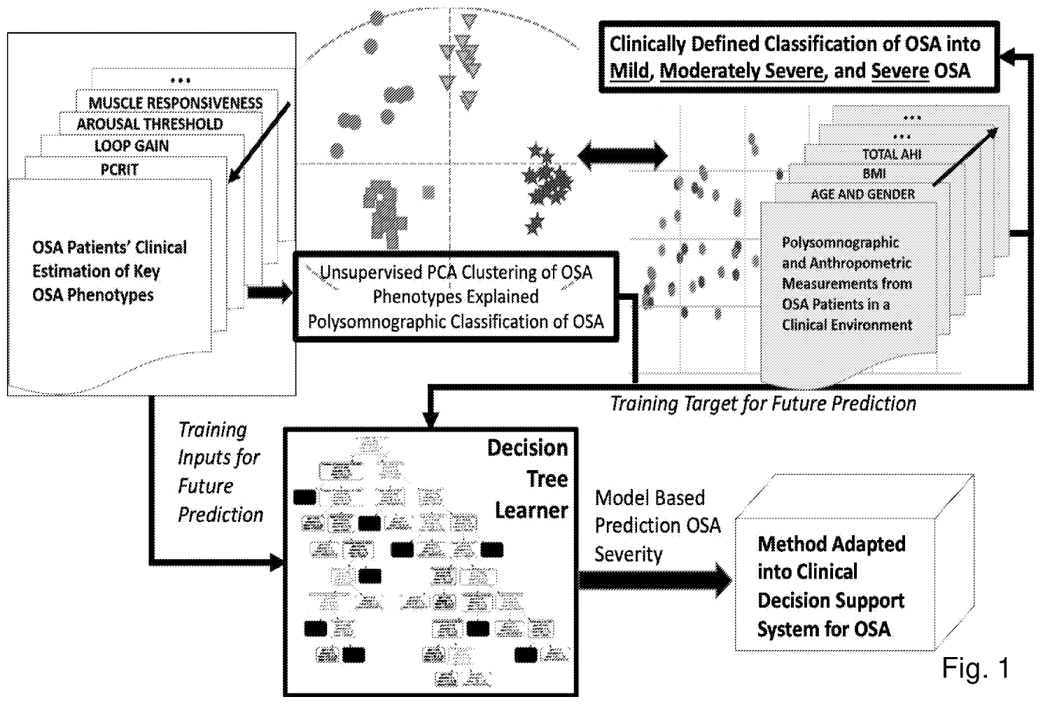

### DETAILED DESCRIPTION

The embodiments are directed at determining the extent to which standard polysomnographic and anthropometric variables can be used to accurately estimate key OSA phenotypes/endotypes including: 1) upper-airway collapsibility (Pcrit), 2) arousal threshold, 3) loop gain and 4) pharyngeal muscle responsiveness. The four pathophysiogical phenotypes/endotypes, namely, Pcrit, Arousal threshold, Loop gain, and Muscle responsiveness have recently been identified as contributors to OSA pathogenesis and may provide new targets for therapy.

The PALM scale categorizes patients into three levels of OSA severities based on the level of anatomical compromise determined via Pcrit, namely, GOOD (Pcrit <−2cmH₂O), MODERATE (Pcrit between −2 and +2 cmH₂O) and BAD (Pcrit >+2cmH₂O).

The embodiments proceed by collection of age, BMI plus standard polysomnography parameters from a diagnostic study (without CPAP). The 4 key OSA phenotypes were quantified in a cohort of over 50 people with and without OSA of varying severity using gold-standard upper-physiology methodology on a separate night. Unsupervised multivariate principal component analyses (PCA) and data driven supervised machine learning (decision tree learner: DTL) were used to analyze the collected data.

Using this approach standard polysomnographic variables plus age and BMI predicted Pcrit (total AHI was the most influential variable) with 82% accuracy (sensitivity 95% and specificity 87%), arousal threshold (non-REM AHI was the most influential variable) with 83% accuracy (sensitivity 80% and specificity 89%), loop gain (supine non-REM AHI was the most influential variable) with 74% accuracy (sensitivity 92% and specificity 85%) and muscle responsiveness (REM AHI was the most influential variable) with 73% accuracy (sensitivity 85% and specificity 94%).

These findings highlight the potential for routine sleep study and clinical data to estimate OSA phenotypes/endotypes using data driven predictive analysis as an accurate and reliable clinical decision support system to inform targeted treatment for OSA.

The embodiments found that standard clinical (age and BMI) and polysomnographic information (including: total AHI, supine AHI, nadir SaO2, non-REM AHI, supine non-REM AHI, REM AHI, arousal index, the fraction of hypopneas vs. apneas) can be used to estimate the 4 OSA phenotypes/endotypes with a high degree of accuracy.

The embodiments include a method of determining the extent to which standard polysomnographic and anthropometric variables can be used to accurately estimate key OSA phenotypes.

Participants: Initially, data was acquired from a cohort of 43 people with OSA and 9 people without OSA who were not taking any medications known to affect sleep or breathing Participants had been compliant with continuous positive airway pressure (CPAP) therapy assessed via machine download for at least 3 months prior to enrolment. Anthropometric and sleep parameters for the 52 participants are outlined in Table 1 below. Data are mean±SD, where applicable. n=number of participants, BMI=body-mass index, AHI=apnea-hypopnea index, CPAP=continuous positive airway pressure, REM=rapid eye movement, SaO₂=estimated overnight blood oxygen saturation.

| | All participants | OSA | Controls |
|---|---|---|---|
| | (n = 52) | (n = 43) | (n = 9) |
| Gender (M/F) | 33/19 | 30/13 | 3/6 |
| Age (years) | 45 ± 11 | 47 ± 10 | 39 ± 13 |
| BMI (kg/m²) | 34 ± 7 | 35 ± 6 | 27 ± 4 |
| Total AHI (events/h) | 36 ± 30 | 43 ± 30 | 4 ± 3 |
| Supine AHI (events/h) | 37 ± 31 | 44 ± 29 | 4 ± 3 |
| Nadir SaO₂ (%) | 83 ± 7 | 82 ± 7 | 89 ± 3 |
| Non-REM AHI (events/h) | 35 ± 32 | 42 ± 31 | 3 ± 4 |
| Supine non-REM AHI (events/h) | 37 ± 33 | 44 ± 32 | 3 ± 4 |
| REM AHI (events/h sleep) | 37 ± 25 | 42 ± 24 | 13 ± 10 |
| Arousal index (#/h sleep) | 37 ± 27 | 42 ± 27 | 14 ± 6 |
| Fraction of hypopneas: apneas | 86 ± 17 | 85 ± 19 | 90 ± 9 |
| CPAP compliance (h/night) | | 6.3 ± 1.4 | |

Experimental Design and Measurements

Participants completed a detailed sleep physiology study. Electroencephalograms (EEGs; C3-A2/O2-A1), electroculograms (EOGs), and chin electromyogram (EMG) were acquired for sleep stage and arousal scoring. Genioglossus EMG (EMGgg) was measured using fine-wire intramuscular electrodes (Cooner Wire Company, Chatsworth, Calif.) (11). Epiglottic pressure was acquired using a transducer-tipped pressure catheter (Millar Instruments, Houston, Tex.) (11). A nasal CPAP mask was fitted and pressure/airflow measured with pressure transducers (Validyne Corporation, Northbridge, Calif.) and pneumotachograph (Hans Rudolf Inc., Kansas City, Mo.) (2).

Protocol: Initially, a baseline polysomnogram without-CPAP was performed to quantify key polysomnographic measures of OSA severity including total AHI, non-REM AHI, REM AHI, arousal index and supine AHI in each sleep stage, fraction of hypopneas vs. apneas and measures of oxygen saturation. Participants were then studied supine on CPAP sufficient to eliminate inspiratory flow limitation on a separate night. Transient reductions in CPAP were applied for ≤3 minutes during stable non-REM (NREM) sleep to cause varying degrees of upper airway collapse to quantify the four OSA phenotypes (2).

Data and statistical analyses approaches: Age and BMI plus standard polysomnography measures of OSA severity were used for phenotype outcomes analyses and predication outcome analyses respectively. Briefly, unsupervised multivariate principal component analyses (PCA) and data driven supervised machine learning using a decision tree learner (DTL) were used. The possibility to develop a predictive algorithmic framework capable of learning OSA phenotypes from existing clinical data and predict for unknown patients with high accuracy requires an ensemble of methods. Unsupervised data analysis techniques coupled with supervised predictive modelling was chosen as a state-of-the-art methodology for supporting clinical evaluation and satisfying decision making objectives.

Results

Polysomnography and Anthropometric Parameters Significantly Explained by OSA Phenotypes/Endotypes.

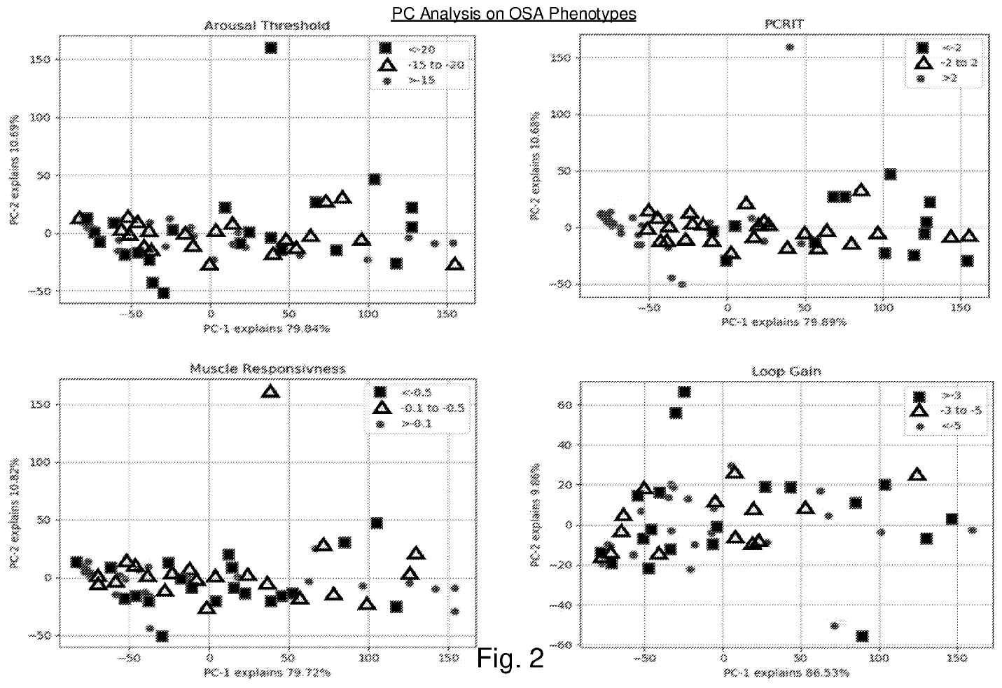

In FIG. 2 four individual unsupervised PCA plots are included representing four PCA scatter plots using patients' polysomnography parameters. Intensity coding (good, moderate, and bad for each trait) is done according to three-class physiological threshold-based definitions of OSA phenotype s/endotypes.

These definitions are summarized in Table 2. Three-class physiological threshold definitions of OSA phenotypes:

| | | | | Muscle |
|---|---|---|---|---|
| | | Loop | Arousal | Responsiveness |
| Class | Pcrit | gain | threshold | (% Max |
| definition | (cmH₂O) | (dimensionless) | (cmH₂O) | EMG/−cmH₂O) |
| GOOD | <−2 | >−3 | <−20 | <−0.5 |
| MODERATE | −2 to +2 | −3 to −5 | −15 to −20 | −0.1 to −0.5 |
| BAD | >2 | <−5 | >−15 | >−0.1 |

% Max EMG/−cmH₂O = Genioglossus electromyography as a percent of maximal activation during wakefulness per cmH₂O of negative epiglottic pressure during sleep. Definitions were defined according to previously established cut-offs (2).

OSA Phenotypes were Important Contributors to Polysomnographic Defined Measures of OSA Severity.

As an additional cross validation the developed predictive model was used to determine if it could be used to accurately classify the presence or absence of OSA and, if OSA was present, its severity level (i.e. no OSA=AHI<10, mild-moderately severe OSA=AHI 10-30 and severe OSA=AHI>30 events/h) using the gold standard physiological measurements of the 4 pathophysiological phenotypes that contribute to OSA.

Principal component analyses using the 4 OSA phenotypes showed linear separability with the first two components. This enabled development of the model to classify different levels of OSA severity.

These observations were separated into three individual sets (total AHI, non-REM AHI, REM AHI) where the most influential contributing OSA phenotype was also identified for each of polysomnography parameter. OSA phenotypes were used to train a supervised DTL model to evaluate the efficiency of a proposed predictive model to explain and predict polysomnography parameters. AHI based OSA severity (or absence of OSA) were classified into three categories, namely, No OSA, Mild-Moderately Severe OSA, and Severe OSA.

It was found that OSA phenotypic traits were important contributors to polysomnographic defined measures of OSA severity. PCA combined with DTL using the 4 OSA phenotypes predicted total AHI (loop gain was the most influential trait) with 87% accuracy (91% sensitivity and 88% specificity), non-REM AHI (Pcrit was the most influential trait) with 90% accuracy (85% sensitivity and 95% specificity) and REM AHI (Arousal threshold was the most influential trait) with 82% accuracy (87% sensitivity and 90% specificity).

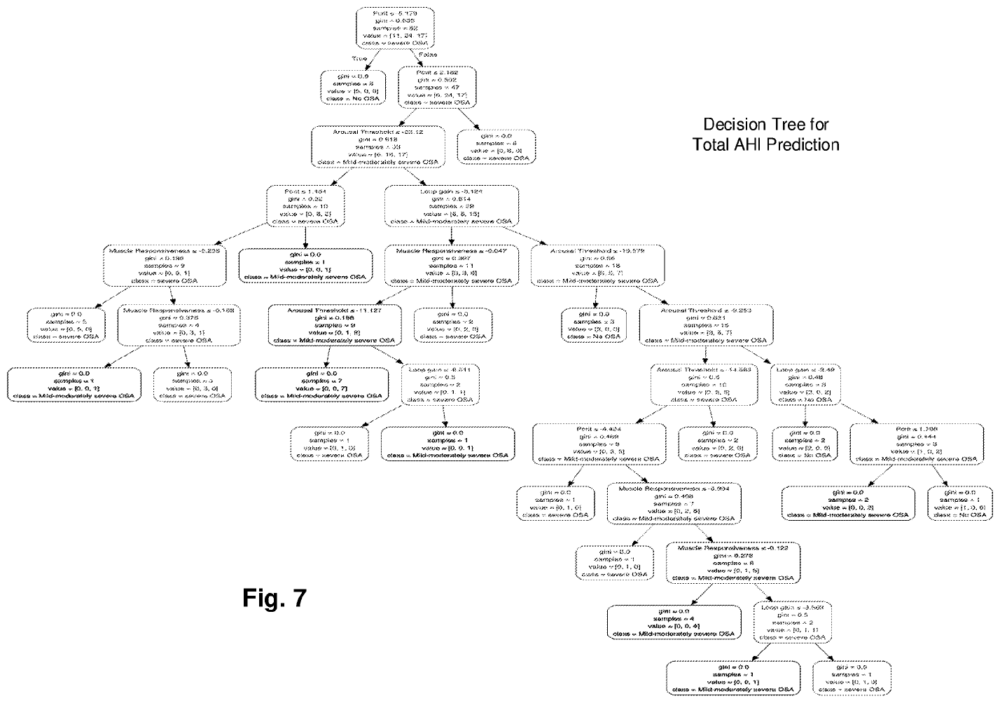

FIG. 7 shows the final decision tree for Total AHI prediction.

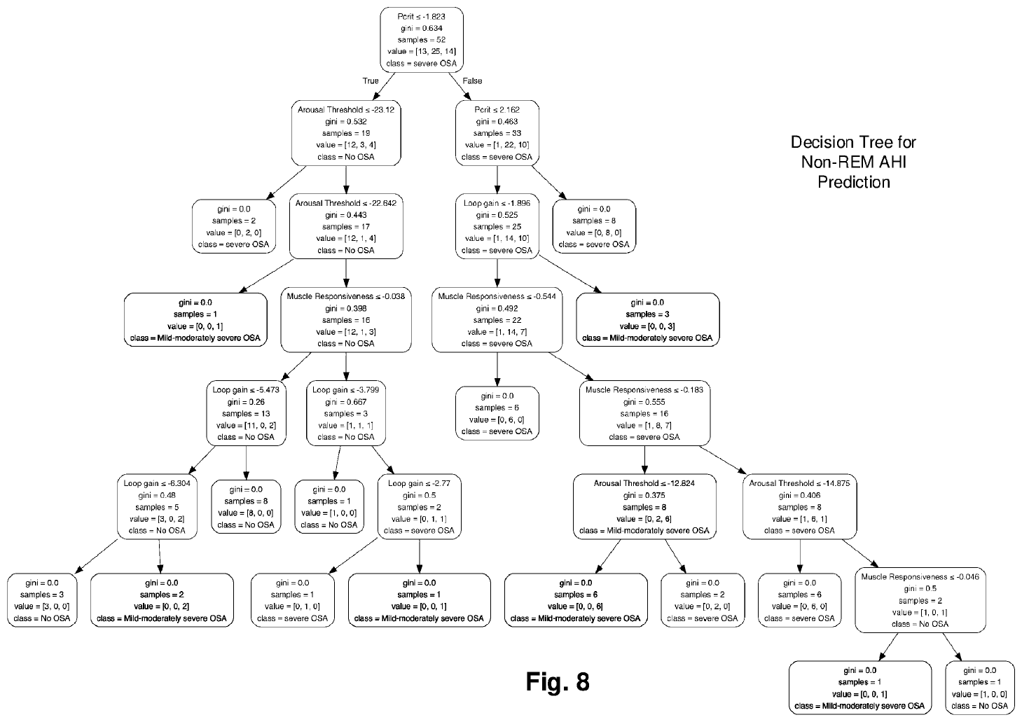

FIG. 8 shows the final decision tree for non-REM AHI prediction.

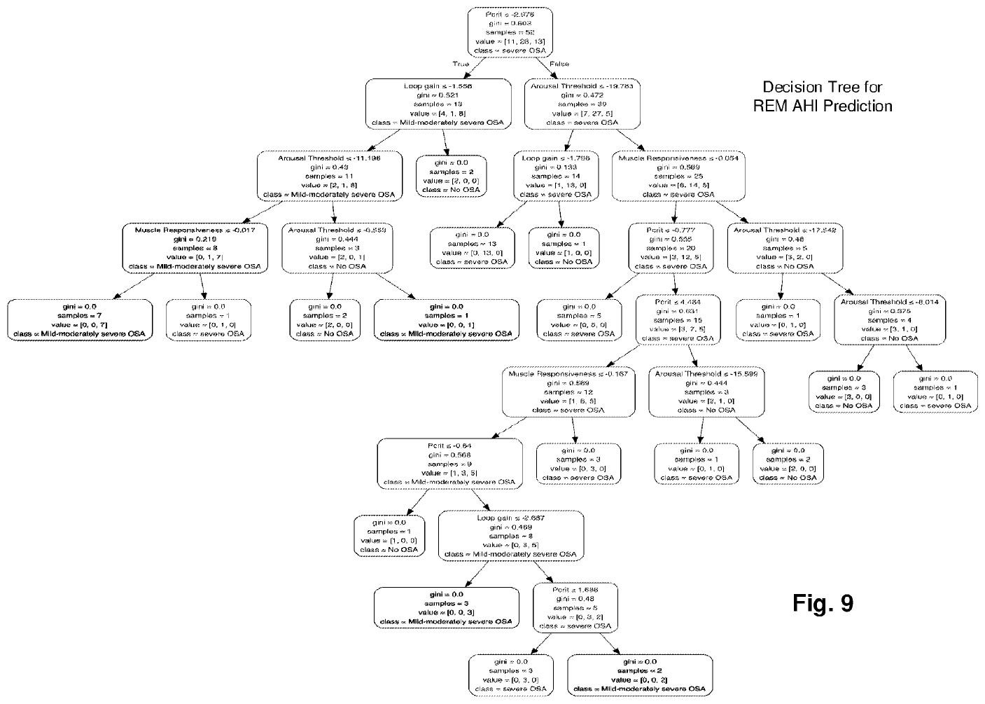

FIG. 9 shows the final decision tree for REM AHI prediction.

Understanding the contribution of OSA pathophysiological phenotypes/endotypes to polysomnography defined parameters of OSA severity is a complex quantification and clinically relevant question. The aim of this part of the method was to quantify the effectiveness of using independently measured OSA phenotypes to explain variability in important polysomnography parameters of OSA severity. Variation explained by PCs provided a clear picture on capabilities and contribution of individual OSA phenotypes while correlating to individual polysomnography parameters. This is only possible as PCA removed all highly correlated variables to refine the data representation purely based on less correlated variables.

Simplified Clinical Tools and PCA Combined with DTL to Predict Treatment Responses to Non-CPAP Therapies for OSA.

The clinical tools and PCA combined with DTL were further utilised to predict treatment responses to mandibular advancement therapy and combination therapy with mandibular advancement therapy plus expiratory positive airway pressure (EPAP) valves.

In addition, tests were conducted to conduct comparative modelling using a range of algorithms (i.e. Nearest Neighbors, SVM, Gaussian Process, Decision Tree, Random Forest, Perceptron Neural Net, AdaBoost, Naive Bayes, QDA, Extra Tree, Gradient Boosting and Logistic Regression) to optimise the predictive accuracy and tailor the treatment prediction solution for each intervention/outcome for superior representation.

The goal of the first analysis was to determine if the combination of a total of 12 clinical (Age and BMI) and standard polysomnographic variables from a sleep study off therapy (total AHI, nadir overnight oxygen saturation, supine NREM AHI, sleep efficiency and arousal index) plus simple breathing parameters collected during 5 minutes of quiet breathing awake in the supine position (breathing frequency (Fb), tidal volume (Vt), minute ventilation (Vi), peak inspiratory airflow (PIF) and nasal resistance) could be used to predict treatment outcome (and if so, which ones and to what extent) according to the 4 common definitions of responder vs. non-responder to a novel mandibular advancement device (Oventus O₂Vent®). Studies were also conducted on the accuracy of the clinical and polysomnographic variables (7 in total) alone and the wakefulness breathing variables alone (5 in total) to predict MAS treatment outcome.

This study was performed in 44 people (9 female) with OSA (mean AHI=28±21 events/h sleep) who were on average overweight (BMI=29±4 kg/m²) aged 29-78 years who were studied overnight before and after mandibular advancement therapy with the Oventus device. The 4 definitions of treatment success were: 1) treatment AHI <5 events/h sleep, 2) treatment AHI <10 events/h sleep, 3) treatment AHI >50% reduction from baseline and 4) treatment AHI >50% reduction from baseline to <20 events/h sleep.

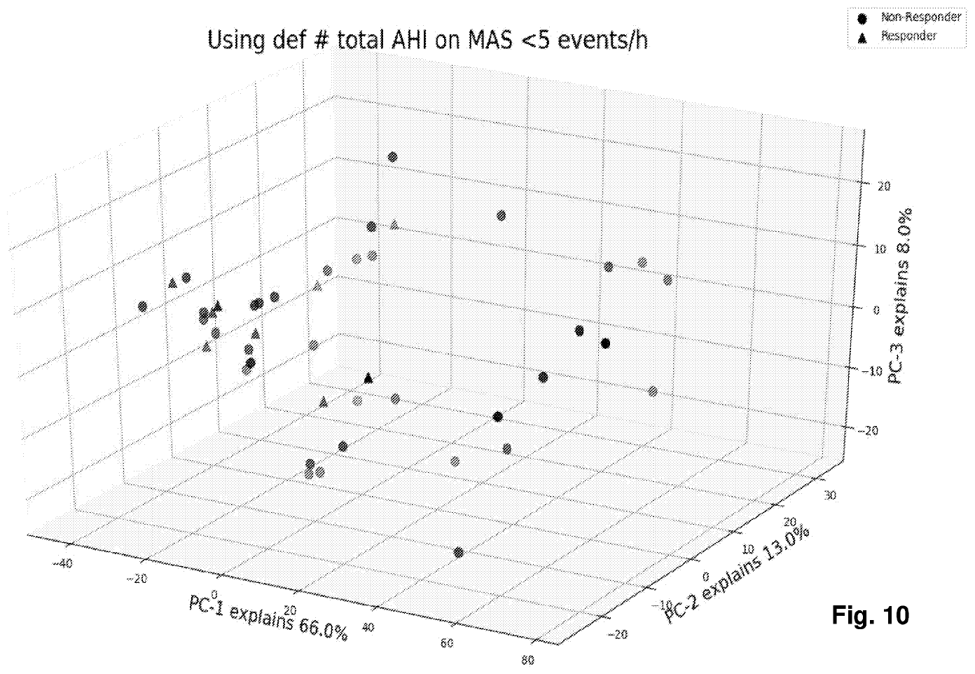

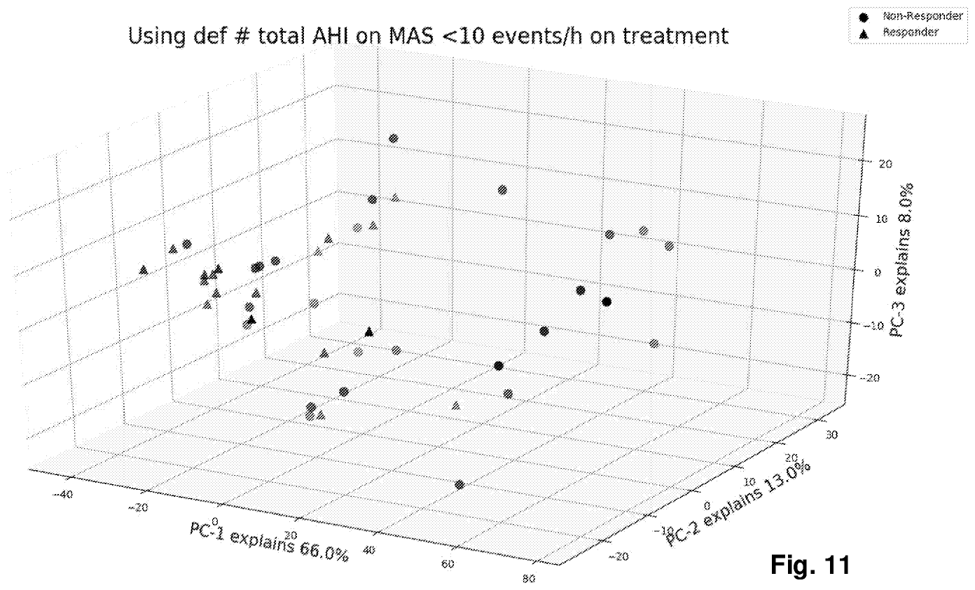

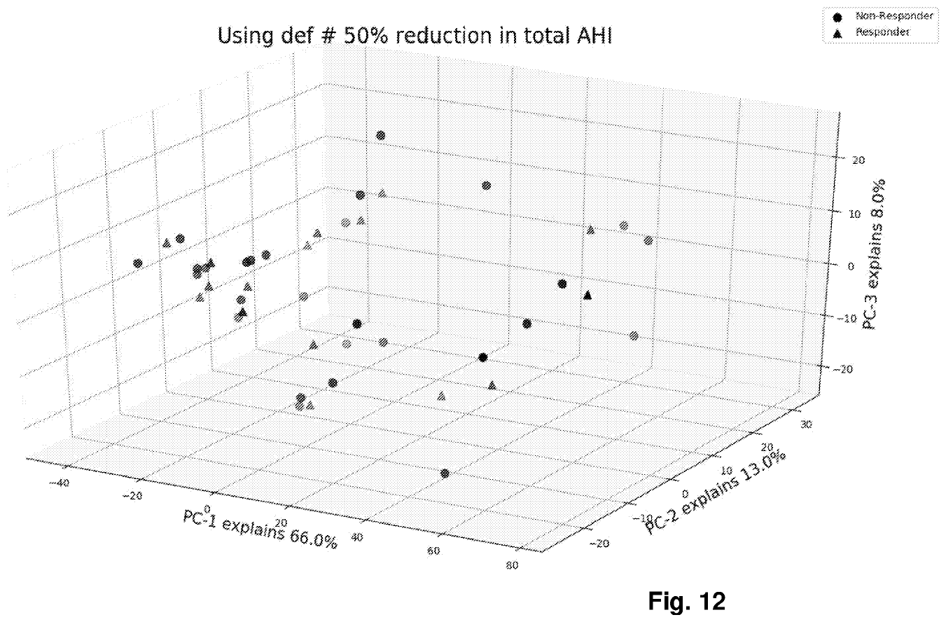

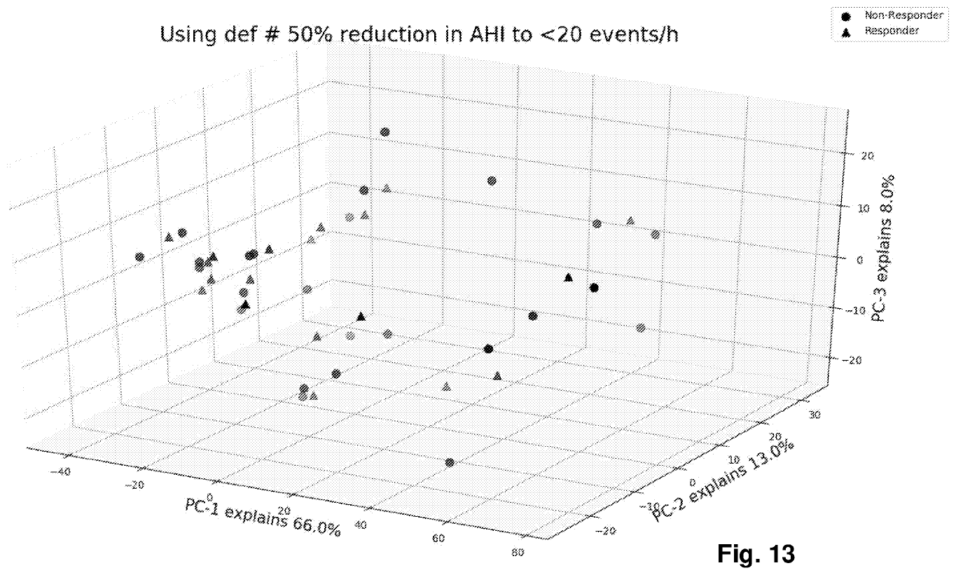

PC analysis explained 87% of information variation using first three PCs derived from the 12 clinical, anthropometric, and breathing inputs. 3D visual variations of the first three PCs are scatter plotted and labelled using four definitions for response classification of ‘Non-Responder’ against ‘Responder’ class (FIGS. 10-13).

Def 1: Explaining “Total AHI on MAS <5 Events/h” Using 12 Clinical, Anthropometric, and Breathing Inputs

Prediction Mean Accuracy: 77% when 10-fold validation was conducted with 50%-50% train-test split with a “Decision Tree algorithm” and 91% with “SVM”. Significance Analysis: “AHI (off MAS)” and “BMI” being the most influential contributors in predicting a response class (treatment outcome).

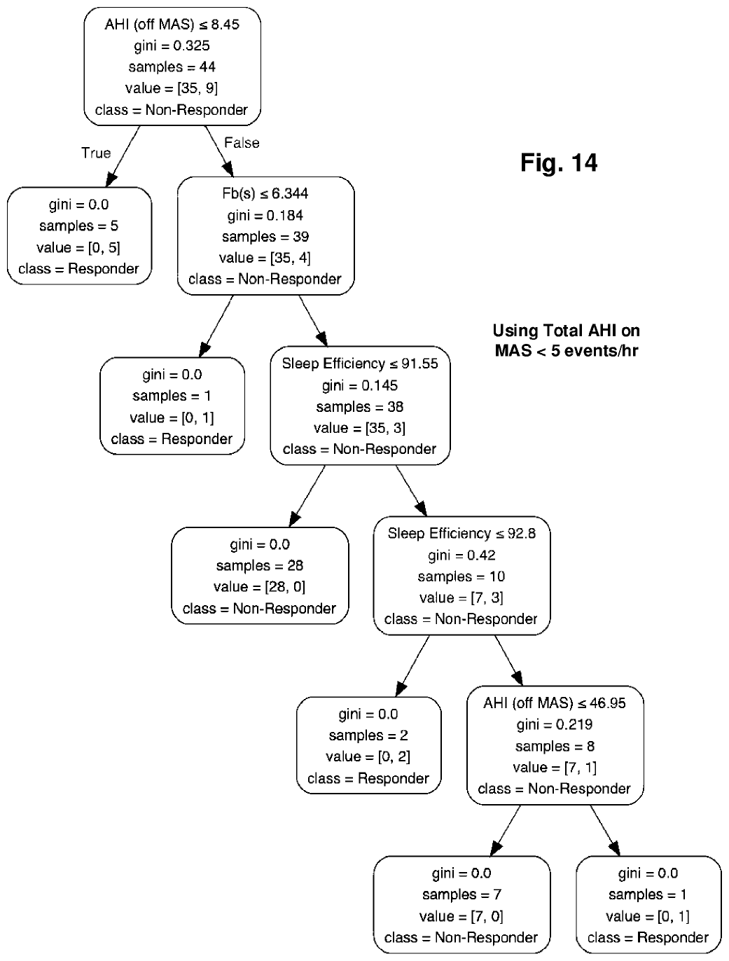

Visualisation of the trained Tree is illustrated in FIG. 14: Definition of ‘gini’=Gini impurity is a measure of how often a randomly chosen element from the set would be incorrectly labeled if it was randomly labeled according to the distribution of labels in the subset. It reaches its minimum (zero) when all cases in the node fall into a single target category. At that stage ‘root node’ converge into a ‘leaf node’ demarking end of a decision branch, or ‘root node’ itself become a ‘leaf node’ with ‘gini=0’.

Def 2: Explaining “Total AHI on MAS <10 Events/h on Treatment” Using 12 Clinical, Anthropometric, and Breathing Inputs

Prediction Mean Accuracy was 73% when 10 fold validation was conducted with 50%-50% train-test split with a “Decision Tree algorithm” and 82% with “Naive Bayes”. Significance Analysis: “minute ventilation” and “Supine NREM AHI (off MAS)” being the most influential contributors in predicting a response class (treatment outcome).

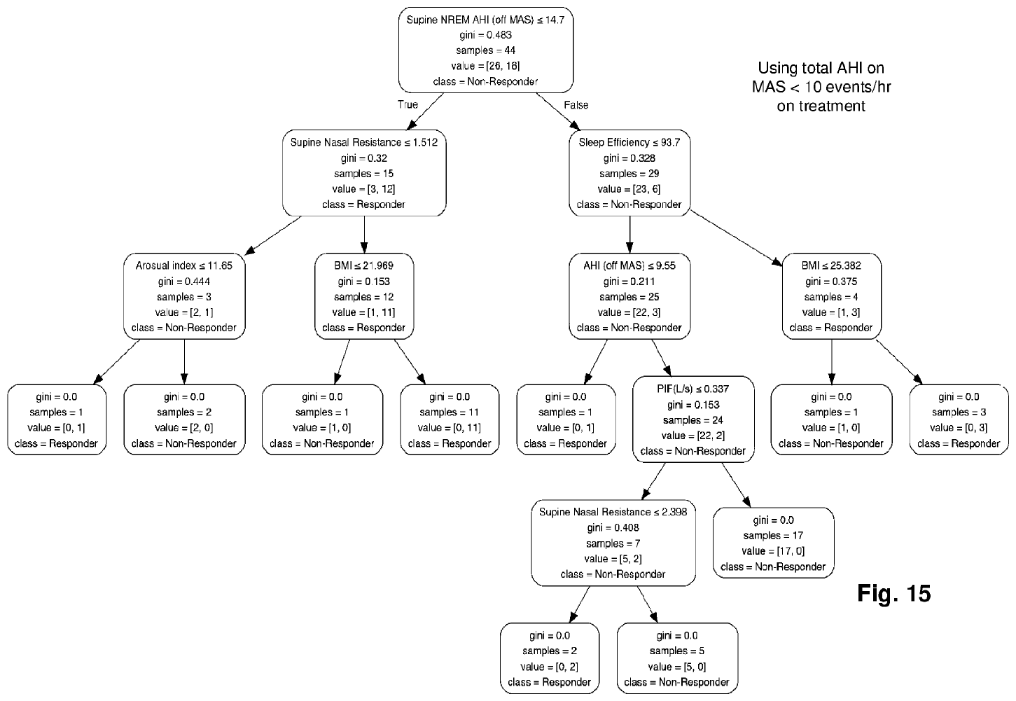

FIG. 15 illustrates a visualisation of the resultant trained Tree.

Def 3: Explaining “50% Reduction in Total AHI” Using 11 Clinical, Anthropometric, and Breathing Inputs

Prediction Mean Accuracy was 68% when 10 fold validation was conducted with 50%-50% train-test split with a Decision Tree algorithm and 77% with “AdaBoost”. Significance Analysis: “Nadir SaO₂ (off MAS)” and “tidal volume” being the most influential contributors in predicting a response class (treatment outcome).

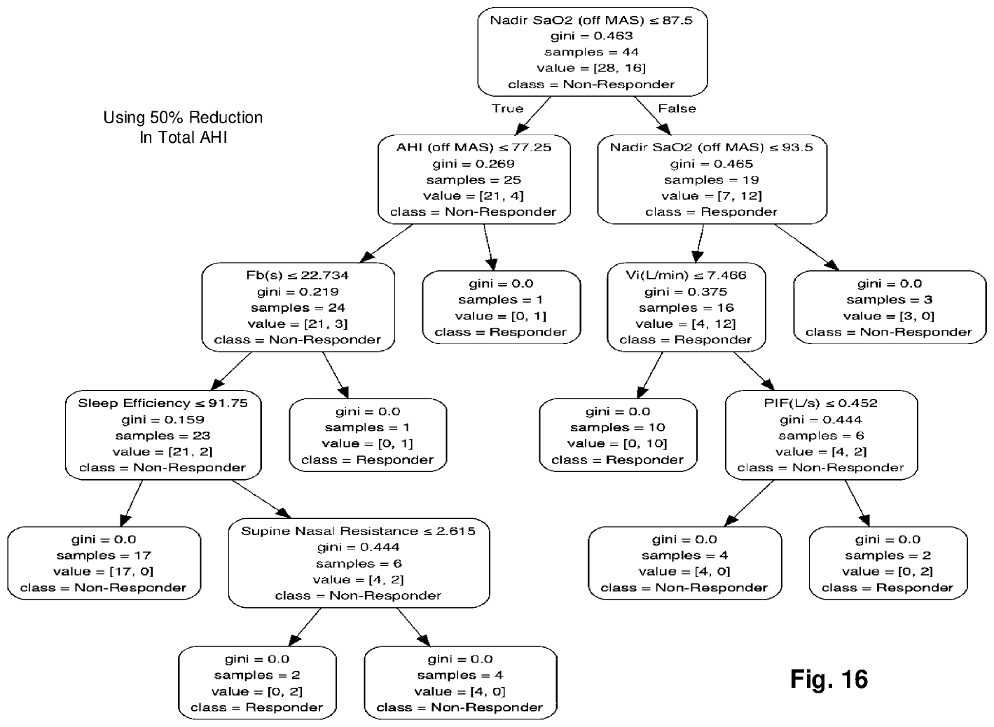

FIG. 16 illustrates a visualisation of the resultant trained Tree.

Def 4: Explaining “50% Reduction in AHI to <20 Events/h” Using 12 Clinical, Anthropometric, and Breathing Inputs

Prediction Mean Accuracy was 64% when 10 fold validation was conducted with 50%-50% train-test split with a Decision Tree algorithm and 73% with “AdaBoost”. Significance Analysis: “Nadir SaO₂ (off MAS)” and “breathing frequency” being the most influential contributors in predicting a response class (treatment outcome).

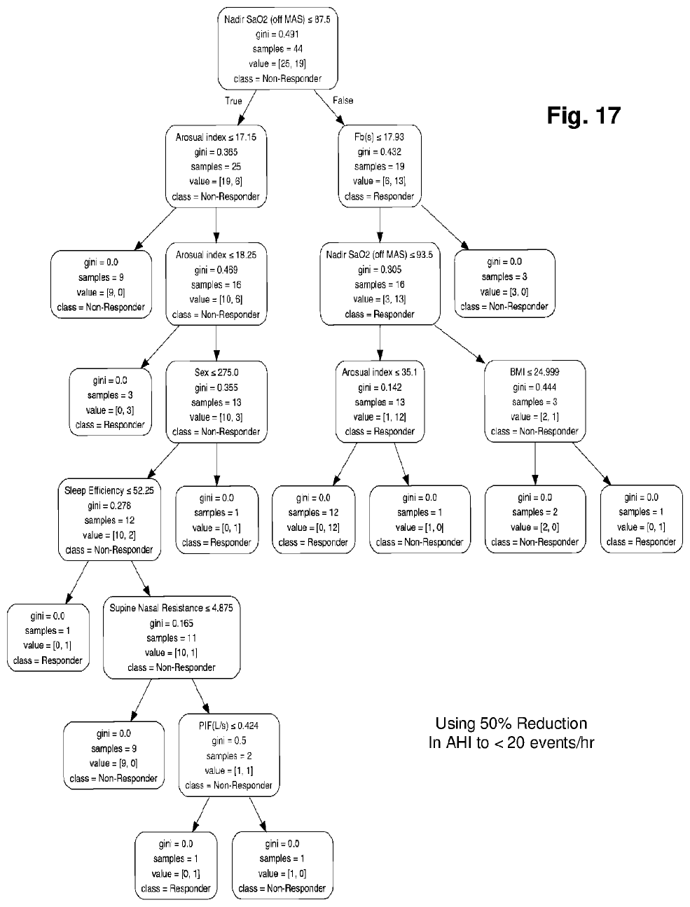

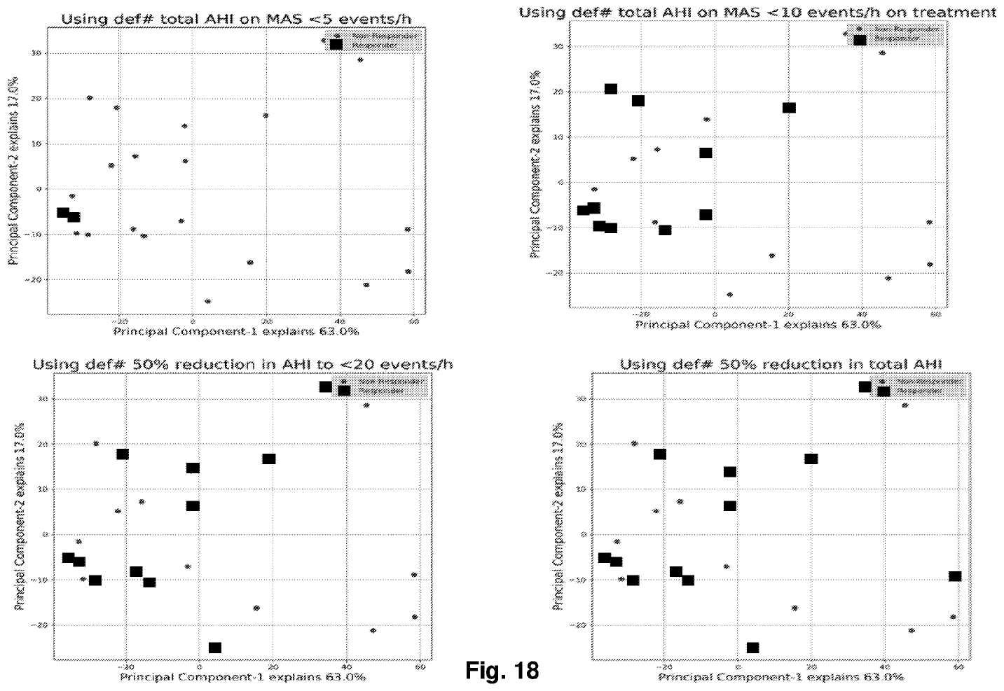

FIG. 17 illustrates a visualisation of the resultant trained Tree.

As a prospective validation and benchmarking investigation, the optimized algorithm was used with the same 12 clinical, anthropometric, and breathing inputs in 17 new patients with OSA who were studied overnight on MAS therapy with an Oventus device to test the accuracy of the previously trained algorithm to predict treatment responders according to Def 1: “total AHI on MAS <5 events/h”. This analysis was performed blinded to the treatment outcome.

The 17 participants were aged between 30-65 years, two were female, on average they had moderately severe OSA (mean AHI=23±12, range=10-55 events/h sleep) and on average were overweight (mean BMI=28±4, range=23-35 kg/m²)

The model successfully predicted the treatment outcome in all 17 participants (100% accuracy) in this prospective cohort.

The model was also tested using a total of 7 clinical and anthropometric inputs in the 44 participants from the first MAS therapy trial.

Def 1: Explaining “Total AHI on MAS <5 Events/h” Using a Total of 7 Clinical and Anthropometric Inputs.

Prediction Mean Accuracy: 86% when 10-fold validation was conducted with 50%-50% train-test split with a “Decision Tree algorithm” and 91% with “Gaussian Process”. Significance Analysis: “AHI (off MAS)” and “BMI” being the most influential contributors in predicting a response class (treatment outcome).

Def 2: Explaining “Total AHI on MAS <10 Events/h on Treatment” Using a Total of 7 Clinical and Anthropometric Inputs

Prediction Mean Accuracy was 73% when 10 fold validation was conducted with 50%-50% train-test split with a “Decision Tree algorithm” and 82% with “Naive Bayes”. Significance Analysis: “Supine NREM AHI (off MAS)” and “AHI (off MAS)” being the most influential contributors in predicting a response class (treatment outcome).

Def 3: Explaining “50% Reduction in Total AHI” Using a Total of 7 Clinical and Anthropometric Inputs

Prediction Mean Accuracy was 73% when 10 fold validation was conducted with 50%-50% train-test split with a Decision Tree algorithm and 82% with “Perceptron Neural Net”. Significance Analysis: “Nadir SaO₂ (off MAS)” and “age” being the most influential contributors in predicting a response class (treatment outcome).

Def 4: Explaining “50% Reduction in AHI to <20 Events/h” Using a Total of 7 Clinical and Anthropometric Inputs

Prediction Mean Accuracy was 64% when 10 fold validation was conducted with 50%-50% train-test split with a Decision Tree algorithm and 73% with “AdaBoost”. Significance Analysis: “Nadir SaO₂ (off MAS)” and “BMI” being the most influential contributors in predicting a response class (treatment outcome).

The model was also tested using 5 breathing inputs in the 44 participants from the first MAS therapy trial.

Def 1: Explaining “Total AHI on MAS <5 Events/h” Using 5 Breathing Inputs

Prediction Mean Accuracy was 82% when 10-fold validation was conducted with 50%-50% train-test split with a “Decision Tree algorithm” and 91% with “Naive Bayes”. Significance Analysis: “PIF” and “Nasal Resistance” being the most influential contributors in predicting a response class (treatment outcome).

Def 2: Explaining “Total AHI on MAS <10 Events/h on Treatment” Using 5 Breathing Inputs

Prediction Mean Accuracy was 77% when 10 fold validation was conducted with 50%-50% train-test split with a “Decision Tree algorithm” and 82% with “Naive Bayes”. Significance Analysis: “minute ventilation” and “Nasal Resistance” being the most influential contributors in predicting a response class (treatment outcome).

Def 3: Explaining “50% Reduction in Total AHI” Using 5 Breathing Inputs

Prediction Mean Accuracy was 68% when 10 fold validation was conducted with 50%-50% train-test split with a Decision Tree algorithm and 82% with “Perceptron Neural Net”. Significance Analysis: with “minute ventilation” and “tidal volume” being the most influential contributors in predicting a response class (treatment outcome).

Def 4: Explaining “50% Reduction in AHI to <20 Events/h” Using 5 Breathing Inputs

Prediction Mean Accuracy was 64% when 10 fold validation was conducted with 50%-50% train-test split with a Decision Tree algorithm and 73% with “AdaBoost”. Significance Analysis: “minute ventilation” and “Nasal resistance” being the most influential contributors in predicting a response class (treatment outcome).

The same 10 variables were then used including two clinical (BMI and age) and eight polysomnographic variables (total AHI, supine AHI, nadir SaO₂, non-REM AHI, supine non-REM AHI, REM AHI, arousal index, the fraction of hypopneas vs. apneas) and were included in the phenotype study of 52 participants as a cross-validation for MAS therapy prediction. These analyses were performed in 35 of the 44 participants from the MAS efficacy study in whom all of these 10 variables were available for analysis.

Def 1: Explaining “Total AHI on MAS <5 Events/h” Using a Total of 10 Clinical and Anthropometric Inputs

Prediction Mean Accuracy: 89% when 10-fold validation was conducted with 50%-50% train-test split with a “Decision Tree algorithm” and 94% with “Extra Tree”. Significance Analysis: “total AHI” and “Supine AHI” being the most influential contributors in predicting a response class (treatment outcome).

Def 2: Explaining “Total AHI on MAS <10 Events/h on Treatment” Using a Total of 10 Clinical and Anthropometric Inputs

Prediction Mean Accuracy was 72% when 10 fold validation was conducted with 50%-50% train-test split with a “Decision Tree algorithm” and 78% with “Random Forest”. Significance Analysis: “Supine AHI” and “BMI” being the most influential contributors in predicting a response class (treatment outcome).

Def 3: Explaining “50% Reduction in Total AHI” Using a Total of 10 Clinical and Anthropometric Inputs

Prediction Mean Accuracy: 78% when 10 fold validation was conducted with 50%-50% train-test split with a Decision Tree algorithm and 83% with “Random Forest”. Significance Analysis: “Nadir SaO₂” and “REM AHI” being the most influential contributors in predicting a response class (treatment outcome).

Def 4: Explaining “50% Reduction in AHI to <20 Events/h” Using a Total of 10 Clinical and Anthropometric Inputs

Prediction Mean Accuracy was 67% when 10 fold validation was conducted with 50%-50% train-test split with a Decision Tree algorithm and 72% with “Logistic Regression”. Significance Analysis: “Nadir SaO₂” and “arousal index” being the most influential contributors in predicting a response class (treatment outcome).

In another treatment analysis, 22 people with OSA were studied, who were not responders or were incomplete responders (residual AHI on treatment >10 events/h sleep) to mandibular advancement therapy alone with combination therapy (mandibular advancement therapy plus oral and nasal EPAP therapy) during an overnight sleep study.

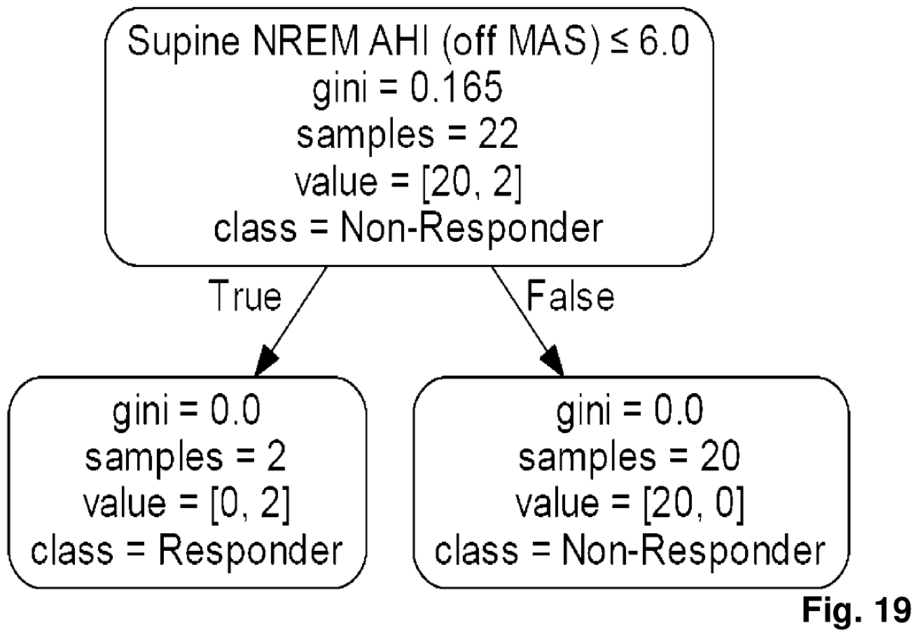

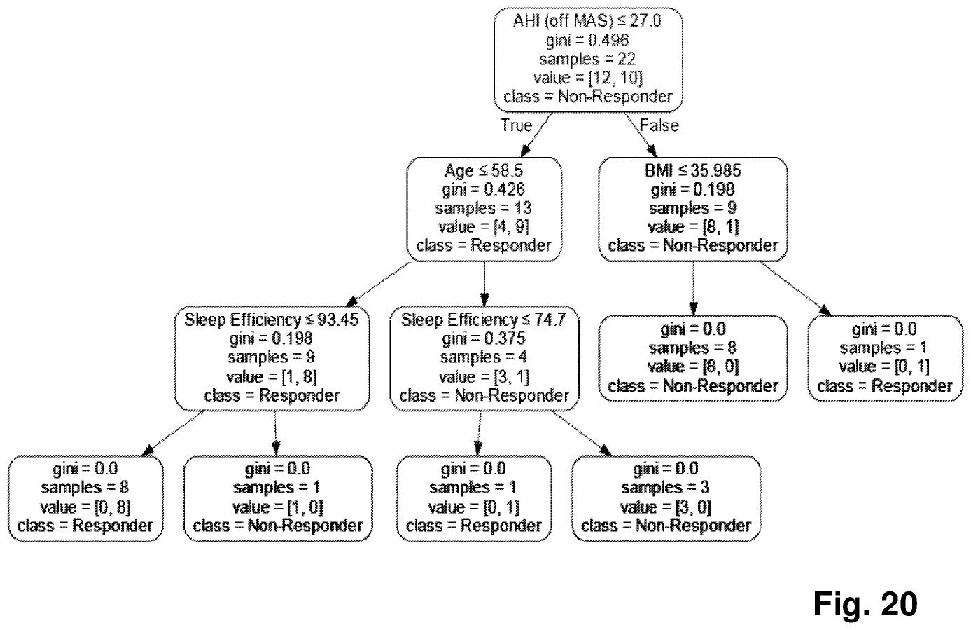

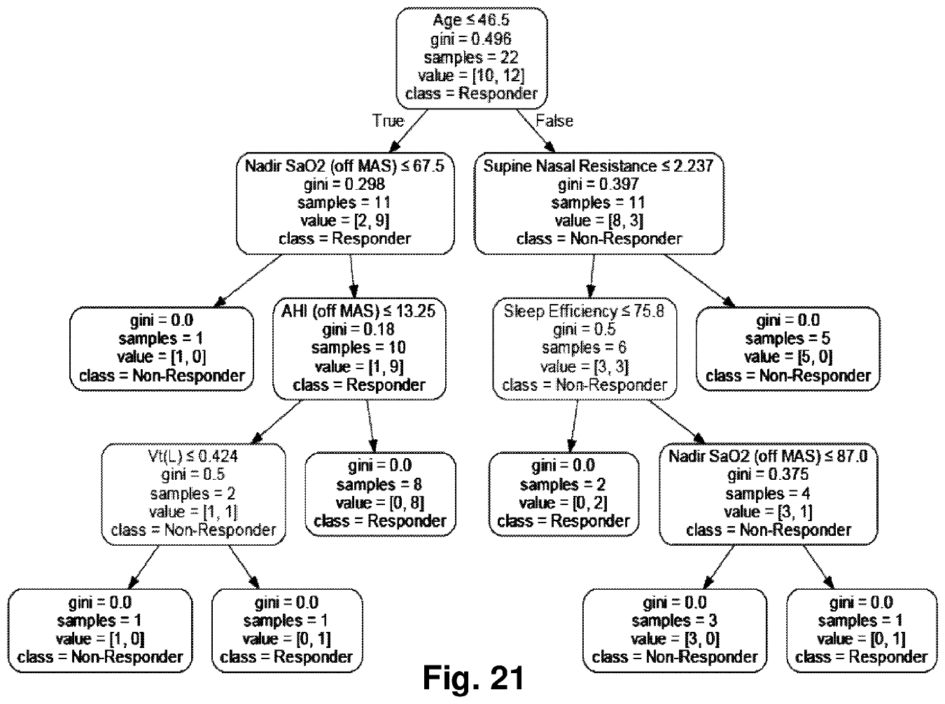

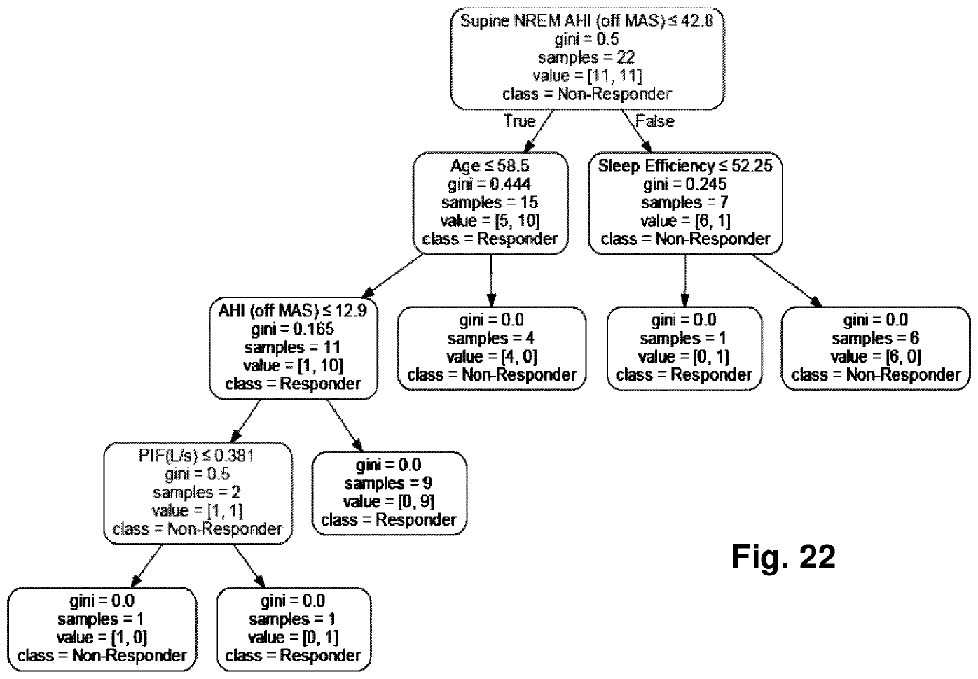

The same 12 clinical and simple breathing parameters were used and analysis tools to predict combination therapy treatment outcome. Results according to the four responder treatment definitions are displayed in FIG. 19-22.

Methods: Multivariate Principal Component Analyses (PCA)

Classical or multivariate statistical methods using a probability model were first developed in the fields of applied mathematics for multi-component data analysis and pattern recognition. Categorization of classifiers can be made depending on certain aspects, such as supervised or unsupervised, model-based or model-free, qualitative or quantitative. For example, discriminate function analysis is a parametric and supervised learning classifier, which can be used for both qualitative and quantitative analysis. PCA is an unsupervised linear non-parametric projection method and is often used to implement a linear supervised classifier, in conjunction with discriminate analysis.

Comparative prediction accuracy: PCA depicted indicated that the majority of the information variations in the polysomnography parameters could be explained by the first two PCs. This process and results strongly indicated indicates that variables were linearly separable. Thus, a suitable data driven supervised predictor could be trained with polysomnography parameter data as training inputs and OSA phenotypes classes as training targets to achieve reasonable prediction accuracy in a clinical context. The measured phenotype traits were incorporated into four individual models combining to predict OSA severity. The model validity is determined by comparing the model prediction of OSA to the clinical diagnosis of OSA. The phenotype data set (n=52) were analysed using five different types of supervised ANN classifiers, namely the Multi Layer Perceptron Network (MLPN), Random Forest Network (RFN), Probabilistic Neural Network (PNN), Radial Basis Function Network (RBFN) and Decision Tree Network (DTN) paradigms. Training of the neural networks was performed with 80% of the whole data set. The remaining 20% of the whole data were used for testing the neural networks. These percentages were selected arbitrarily and were applied for all four of the individual training-testing paradigms. The aim of this comparative study was to identify the most appropriate ANN paradigm, which can be trained with the best accuracy to predict three different levels of OSA severities. Table 2(a), 2(b), 2(c), and 2(d) summarizes the prediction results achieved from the neural networks, using same training inputs and four different OSA severity categories defined by four different phenotype traits (see Table 2).

TABLE 2(a)
Comparative accuracy analysis while trained
using Pcrit based OSA severity categories.

| Prediction Model | Accuracy | Sensitivity | Specificity |
|---|---|---|---|
| MLPN | 60% | 88% | 80% |
| PNN | 72% | 80% | 85% |
| RBFN | 76% | 90% | 92% |
| RFN | 79% | 85% | 90% |
| DTN | 83% | 92% | 89% |

TABLE 2(b)
Comparative accuracy analysis while trained using
Loop gain based OSA severity categories.

| Prediction Model | Accuracy | Sensitivity | Specificity |
|---|---|---|---|
| MLPN | 70% | 78% | 83% |
| PNN | 62% | 88% | 86% |
| RBFN | 78% | 85% | 90% |
| RFN | 79% | 81% | 86% |
| DTN | 80% | 94% | 91% |

TABLE 2(c)
Comparative accuracy analysis while trained using
Arousal Threshold based OSA severity categories.

| Prediction Model | Accuracy | Sensitivity | Specificity |
|---|---|---|---|
| MLPN | 50% | 68% | 80% |
| PNN | 62% | 90% | 75% |
| RBFN | 76% | 80% | 72% |
| RFN | 72% | 75% | 90% |
| DTN | 85% | 95% | 92% |

TABLE 2(d)
Comparative accuracy analysis while trained using Muscle
Responsiveness based OSA severity categories.

| Prediction Model | Accuracy | Sensitivity | Specificity |
|---|---|---|---|
| MLPN | 73% | 81% | 70% |
| PNN | 70% | 70% | 75% |
| RBFN | 69% | 80% | 82% |
| RFN | 77% | 85% | 80% |
| DTN | 81% | 90% | 85% |

Reasoning of prediction accuracy: From table 2(a)-2(b), it is evident that there are two main reasons for the superior classification performance of the DTN technique compared to MLPN, PNN, RBFN, and RFN. These reasons are:

DTNs are able to adapt themselves to the distribution in databases where the number of patterns per category is uneven. Thus, while DTN are able to classify most of the patterns corresponding to categories, MLPN, PNN, are RBFN are less able to adapt to the uneven distribution of samples.

DTN and RFN are able to adjust their scale of generalization to match the morphological variability of the patterns. They were able to achieve a better performance than others in separating of the OSA categories were.

In the case of the DTN algorithm, when a relatively very good solution has been found, the situation can be further refined by modifying the boundaries where misclassification occur, and also by collecting more clinical data in future cohort studies.

The embodiments can use a multivariate statistical method, based on the Karhunen-Loeve expansion, used in classification models to produce classification results for pattern recognition techniques. The method consists of expressing the response vectors, rᵢⱼ, in terms of linear combinations of orthogonal vectors, and is sometimes referred to as vector decomposition. Each orthogonal vector principal component, accounts for a certain amount of variance in the data with a decreasing degree of importance. The scalar product of the orthogonal vectors with the response vector gives the value of the pth principal components:

Xₚ=α₁ₚr₁ⱼ+α₂ₚr₂ⱼ+ . . . +aᵢₚrᵢⱼ+ . . . +αₙₚrₙⱼ

The variance of each PC score, Xp, is maximized under the constraint that the sum of the coefficients of the orthogonal vectors or eigenvectors αp=(α1p . . . αjp . . . αnp) is set to unity, and the vectors are uncorrelated. Since there is often a high degree of sensor co-linearity in control point data, the majority of the information held in response space can often be displayed using a small number of PCs. PCA is in essence a data reduction technique for correlated data, such that a n-dimensional problem can be described by a two or three dimensional plot. It can be applied to high dimensional data sets to identify their variation in structure for future classification and optimization.

Supervised Decision Tree Learning (DTL)

In a supervised pattern recognition engine, a set of known input variables are systematically introduced to the learning algorithm, which are then classified according to the known a priori descriptors or classes held in a knowledge base. In the second stage for identification, an unknown sample is tested against the knowledge base and then the most probably class is predicted. Unknown sample vectors are analyzed using relationships found a priori from a set of known sample vectors used in an initial calibration, learning or training stage. The idea of testing using unknown response vectors is a known technique and often referred to as cross-validation (Kohavi, Ron. “A study of cross-validation and bootstrap for accuracy estimation and model selection.” Ijcai. Vol. 14. No. 2. 1995). It is important for the accurate prediction of new samples.

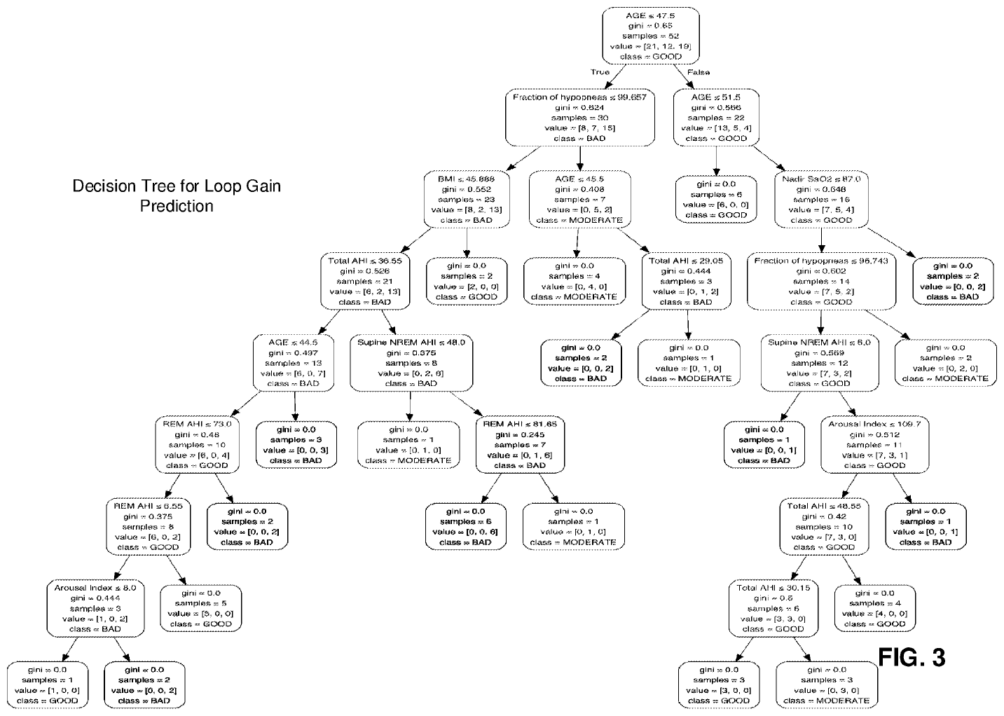

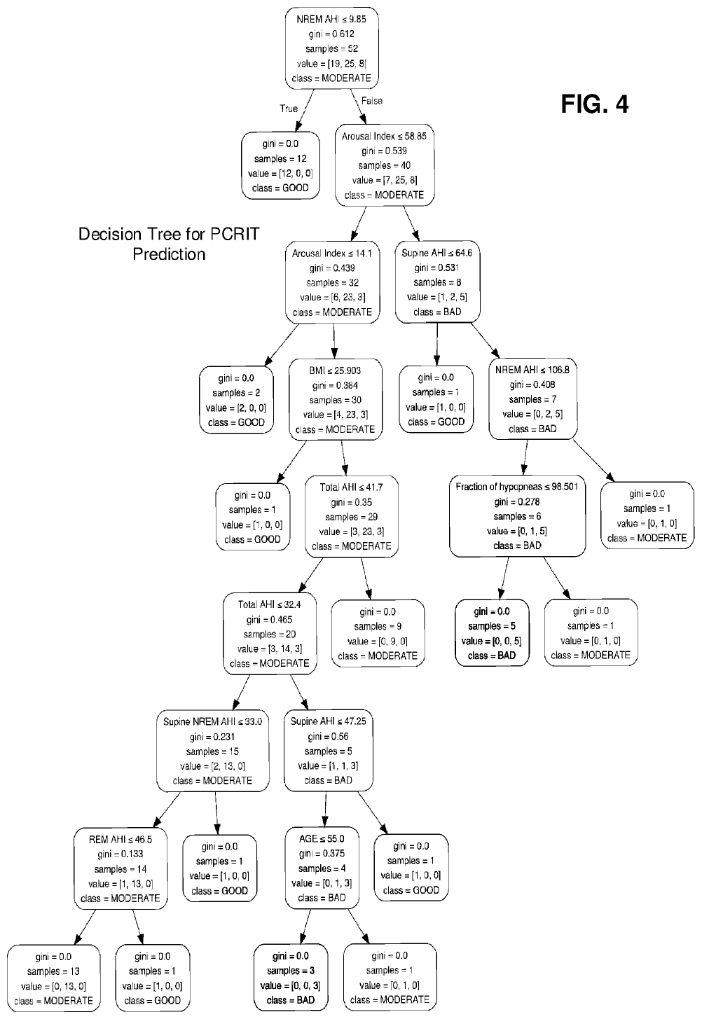

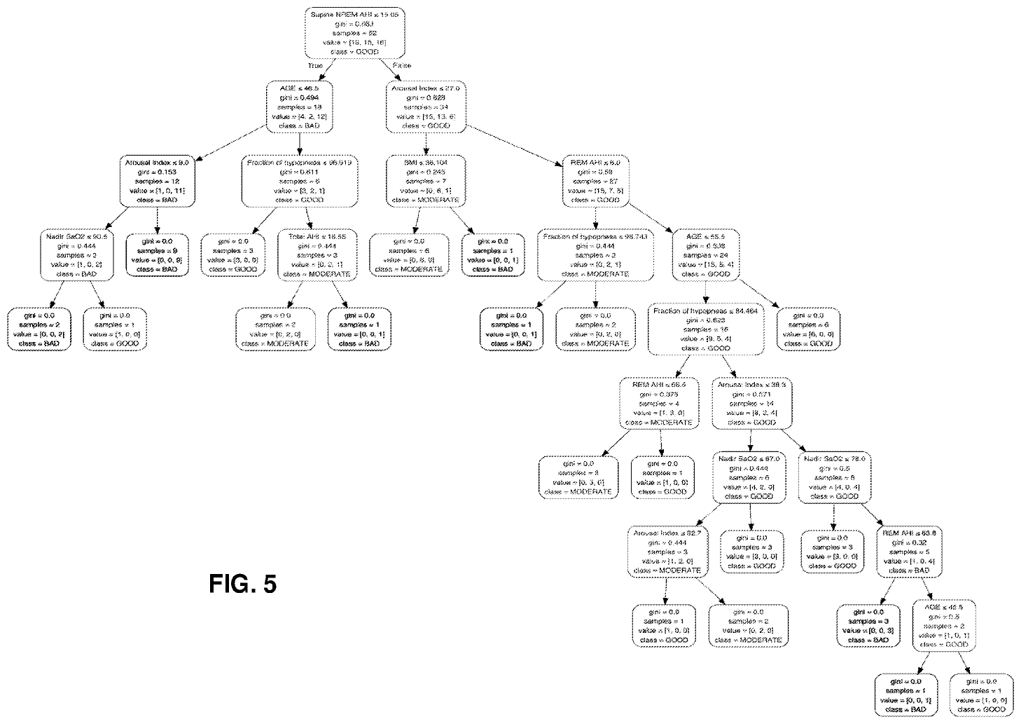

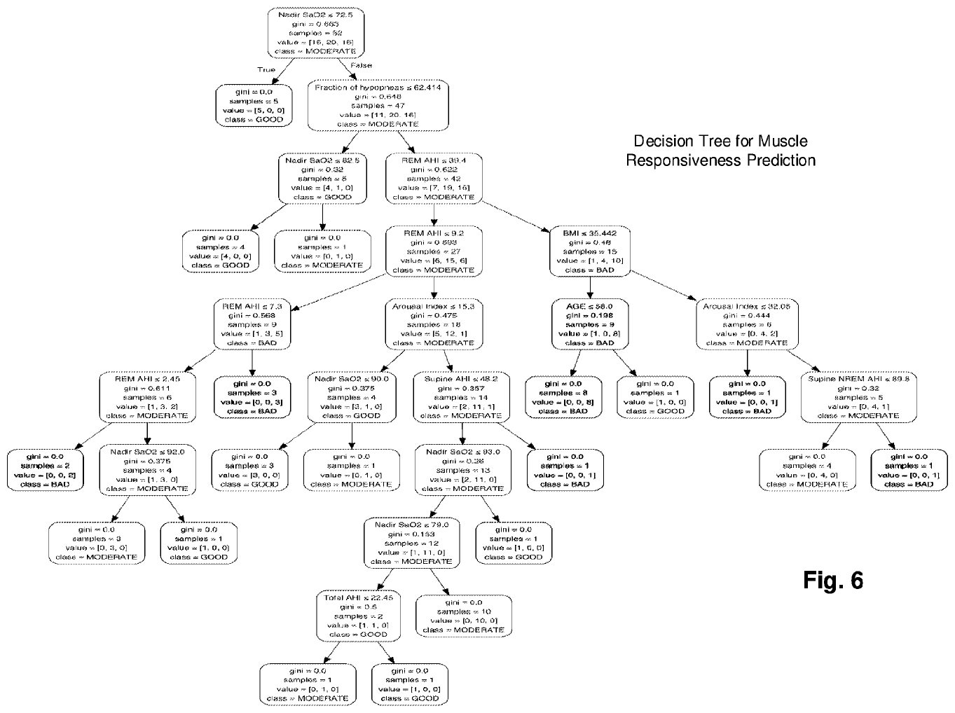

A DTL is a decision support tool that uses a tree-like graph or model of decisions and their possible consequences, including chance event outcomes, resource costs, and utility. It is one way to display an algorithm that only contains conditional control statements. This data driven machine learning approach is very useful to analyse and learn a multivariate clinical data set to formulate a cross-correlation tree like structure to be used to explain underlying relationships among the input variable while predicting a target variable. Prediction from a trained decision tree can be used as a decision support tool with the advantage of knowing “why” that prediction was made by the algorithm based on visually analysis of the trained tree nodes and leaves. FIG. 3 to FIG. 6 illustrates resulting tree like structures. This was an essential criterion for a clinical trial for selecting DTL in the process of predicting OSA phenotypes based on polysomnography parameters.

Predicting the OSA Phenotypes Using Polysomnography Parameters

PCA indicated that using the 4 OSA phenotypes showed linear separability with the first two components. This enabled development of a model to classify different levels of OSA phenotypes. Similarly, the majority of the variation in polysomnography parameters were explained by the first two PCs. This indicates that variables were linearly separable. Thus, a data driven supervised predictor could be trained with polysomnography data as training inputs and OSA phenotypes classes as training targets. Considering the that number of samples are significantly low for a supervised model to be developed effectively to achieve very high accuracy rate, relatively few modelling exercises were conducted to evaluate the capability prediction of a trait class (namely GOOD, MODERATE and BAD class as described in Table 2) using all of each patients' polysomnography parameter data. DTL was trained and tested with various split sets from the whole set. With 80% of samples for training and 20% for testing at a random selection process in a DTL:

Each of the 4 phenotypic traits (Loop gain, Pcrit, Arousal threshold and Muscle responsiveness) could be predicted with a high degree of accuracy (>70%) and high sensitivity and specificity (Table 3 below) (FIGS. 3-6 show the final decision trees for the prediction of each phenotype)

TABLE 3

| Outcome | Accuracy (%) | Sensitivity (%) | Specificity (%) |
|---|---|---|---|
| Loop gain | 74 | 92 | 85 |
| Pcrit | 82 | 95 | 87 |
| Arousal threshold | 88 | 80 | 89 |
| Muscle responsiveness | 73 | 85 | 94 |

These data were generated using a generic model using DTL. The whole learning process within the Decision Tree Predictor was visualised for further clinical analysis and validation of the correct path to prediction. (FIGS. 3-6). Gini impurity (defined as “gini” in the figures) is a measure of how often a randomly chosen element from the set would be incorrectly labeled if it was randomly labeled according to the distribution of labels in the subset. It reaches its minimum (zero) when all cases in the node fall into a single target category. At that stage ‘root node’ converge into a ‘leaf node’ demarking end of a decision branch, or ‘root node’ itself become a ‘leaf node’ with ‘gini=0’. Over time prediction accuracy could be improved with more research on suitability of various models for this type of data sets and with more clinical data.

Results from PCA and DTL summarize that clinically measured polysomnography and anthropometric parameters could be used to identify important OSA phenotypes to enable a predictive model for targeted treatment. This model was further validated by showing that OSA phenotypes can be used to classify OSA severity and simple breathing, clinical and polysomnography measures can be used to identify responders to mandibular advancement therapy and combination therapy in people with OSA.

Interpretation

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

As used herein, the term “exemplary” is used in the sense of providing examples, as opposed to indicating quality. That is, an “exemplary embodiment” is an embodiment provided as an example, as opposed to necessarily being an embodiment of exemplary quality.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A method of determining a likely indicator for obstructive sleep apnea (OSA) of a human subject, the method including the steps of:
(a) Measuring polysomnography and anthropometric parameters of a series of subjects including Gender (M/F), Age (years), BMI (kg/m²), Total AHI (events/h), Supine AHI (events/h), Nadir SaO₂ (%), Non-REM AHI (events/h), Supine non-REM AHI (events/h), REM AHI (events/h sleep), Arousal index (#/h sleep), and Fraction of hypopneas v. apneas.
(b) determining the principal components of the parameters utilising a principal component analysis;
(c) correlating the principal components with the upper-airway collapsibility (Pcrit), arousal threshold, loop gain and pharyngeal muscle responsiveness measurements of each subject;
(d) utilising the correlated measurements to derive a data driven supervised machine learning structure describing the correlation; and
(e) for a given human subject, measuring the subject's Polysomnography and anthropometric parameters and utilising the machine learning structure to determine an estimate of the upper-airway collapsibility, arousal threshold, loop gain and pharyngeal muscle responsiveness of the human subject.

2. A method of predicting the likelihood of responding to one or more obstructive sleep apnea (OSA) treatments for obstructive sleep apnea (OSA) of a candidate subject, the method including the steps of:
(a) measuring a first series of polysomnography and anthropometric parameters for at least a collection of OSA sufferers;
(b) correlating the parameters with at least one of the corresponding upper-airway collapsibility (Pcrit), arousal threshold, loop gain and pharyngeal muscle responsiveness measurement of each subject;
(c) determining a corresponding description structure describing the correlation of step (d); and
(d) utilising the corresponding description structure, in conjunction with a series of polysomnography and anthropometric parameters measured for the candidate subject to predict the likelihood of responding to one or more obstructive sleep apnea (OSA) treatments to thereby provide a clinical decision diagnostic tool for obstructive sleep apnea.

3. A method as claimed in claim 2 wherein said correlating in step (b) includes determining a principal component analysis of the measured parameters of step (a).

4. A method as claimed in claim 3 wherein said correlation in step (b) includes applying multivariate principal component analyses (PCA) to the polysomnography and anthropometric parameters to determine the principal components of the parameters for at least the collection of OSA sufferers.

5. A method as claimed in claim 2 wherein said corresponding descriptive structure comprises a decision tree learner.

6. A method as claimed in claim 5 wherein a decision tree is determined for each of upper-airway collapsibility (Pcrit), arousal threshold, loop gain and pharyngeal muscle responsiveness measurement.

7. A method as claimed in claim 2 wherein said series of polysomnography and anthropometric parameters includes at least one of: age, BMI, total AHI, supine AHI, nadir SaO₂, non-REM AHI, supine non-REM AHI, REM AHI, arousal index, the fraction of hypopneas vs. apneas.

8. A method as claimed in claim 2 wherein the OSA comprises at least one of: 1) upper-airway collapsibility (Pcrit), 2) arousal threshold, 3) loop gain and 4) pharyngeal muscle responsiveness.

9. A method as claimed in claim 2 wherein said step (a) includes measuring OSA sufferers and non-sufferers.

10. A method of determining the likely key phenotypic/endotypic causes of obstructive sleep apnea (OSA), the method including:
(a) measuring a series of polysomnographic and anthropometric variables for the patient with OSA;
(b) predicting the phenotypic/endotypic causes of the OSA for the patient; and
(c) providing predictive information to be used to inform clinical decisions for targeted treatment for the patient based on the predicted causes of the OSA.

11. A method as claimed in claim 10 wherein said step (a) and (b) includes measuring a series of polysomnographic and anthropometric variables for the patient, and using the measured variables to estimate key OSA phenotypes/endotypes for the patient.

12. A method as claimed in claim 10 wherein said step (a) includes measuring OSA phenotypes such as simple breathing, clinical and polysomnography measures, and step (b) includes using the measurements to identify likely responders to mandibular advancement therapy and combination therapy in people with OSA.

13. A method of determining a likely indicator of obstructive sleep apnea (OSA) in a subject patient, the method including the steps of:
(i) for a series of initial patients,
(a) predetermining a first measure of at least one of upper-airway collapsibility (Pcrit), arousal threshold, loop gain and pharyngeal muscle responsiveness;
(b) determining a correlation measure between the first measure and the degree of OSA of the patient.
(ii) for the subject patient:
using the correlation measure and a corresponding first measure for the subject patient to determine a corresponding likely indicator of OSA for the subject patient.

14. A method as claimed in claim 13 wherein said first measure includes a measure of each of upper-airway collapsibility (Pcrit), arousal threshold, loop gain and pharyngeal muscle responsiveness.

15. A method as claimed in claim 13 wherein the correlation is determined via principal component analysis or the other related methods for the first measure factors.

16. A method as claimed in claim 15 wherein the principal component analysis is combined with a decision tree learning system to determine the degree of correlation.

17. A method of predicting responses to one or more targeted therapies to be used as a clinical decision tool for sleep apnea or related respiratory disorders, the method including the steps:
i) Collecting patient information on an individual patient including polysomnographic, anthropometric, demographic, clinical, physiological and breathing variables;
ii) Inputting the patient information into algorithmic tools to predict the accuracy with which the individual patient will respond to a variety of established and emerging treatments including combination therapies;
iii) Presenting the patient information and accuracy prediction to the patient so that they, in combination with their medical provider can make informed decisions about treatment selection;
iv) Retaining and using the collected data from each patient for future use by the algorithmic tools for further refinement and improvement of treatment prediction accuracy on an ongoing basis.
